(19) United States
(12) Patent Application Publication
(10) Pub. No.: US 2024/0208926 A1
(43) Pub. Date: Jun. 27, 2024

(54) NEW PROCESS FOR THE SYNTHESIS OF 5-{5-CHLORO-2-[(3S)-3-[(MORPHOLIN-4-YL)METHYL]-3,4-DIHYDROISOQUINOLINE-2(1H)- CARBONYL]PHENYL}-1,2-DIMETHYL-1H-PYRROLE-3-CARBOXYLIC ACID DERIVATIVES AND ITS APPLICATION FOR THE PRODUCTION OF PHARMACEUTICAL COMPOUNDS

(71) Applicants: LES LABORATOIRES SERVIER, SURESNES CEDEX (FR); NOVARTIS AG, Basel (CH)

(72) Inventors: Frédéric PIN, Bretteville du Grand Caux (FR); Bálint PETHÖ, Érd (HU)

(21) Appl. No.: 18/551,033

(22) PCT Filed: Mar. 23, 2022
(86) PCT No.: PCT/EP2022/057655

§ 371 Date: Sep. 18, 2023

(30) Foreign Application Priority Data
Mar. 24, 2021 (EP) 21305366.3

(51) Int. Cl. C07D 401/10; C07D 207/50; C07K 1/10

(52) U.S. Cl. CPC C07D 401/10; C07D 207/50; C07K 1/10

(57) ABSTRACT

The present invention relates to a new process for preparing 5-{5-chloro-2-[(38)-3-[(morpholin-4-yl)methyl]-3.4-dihydr oisoquinoline-2(1H)-carbonyl]phenyl}-1,2-dimethyl-1/H-pyrrole-3-carboxylic acid derivatives and its application for the production of pharmaceutical compounds.

The present invention relates to a new process for preparing 5-{5-chloro-2-[(3S)-3-[(morpholin-4-yl)methyl]-3,4-dihydroisoquinoline-2(1H)-carbonyl]phenyl }-1,2-dimethyl-1H-pyrrole-3-carboxylic acid derivatives and its application for the production of pharmaceutical compounds.

More specifically, the present invention relates to a new process for preparing ethyl 5-{5-chloro-2-[(3S)-3-[(morpholin-4-yl)methyl]-3,4-dihydroisoquinoline-2(1H)-carbonyl]phenyl}-1,2-dimethyl-1H-pyrrole-3-carboxylate and 5-{5-chloro-2-[(3S)-3-[(morpholin-4-yl)methyl]-3,4-dihydroisoquinoline-2(1H)-carbonyl]phenyl }-1,2-dimethyl-1H-pyrrole-3-carboxylic acid and its application for the production of pharmaceutical compounds.

Even more specifically, the present invention relates to a new process for preparing 5-{5-chloro-2-[(3S)-3-[(morpholin-4-yl)methyl]-3,4-dihydroisoquinoline-2(1H)-carbonyl]phenyl}-1,2-dimethyl-1H-pyrrole-3-carboxylic acid and its application for the production of 5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol- 3-yl)-N-(4-hydroxyphenyl)-1,2-dimethyl-1H-pyrrole-3-carboxamide, referred to herein as ‘Compound A’.

Particularly, the present invention relates to a process for preparing a compound of formula (V):

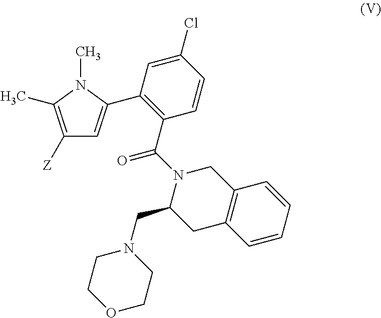

wherein:
Z is a group selected from —COOR and —CN, and
R represents a (C₁-C₆)alkyl group, an allyl group or a —CH₂-aryl group, using a 1,5-dimethyl-1H-pyrrole derivative and a compound of formula (IV):

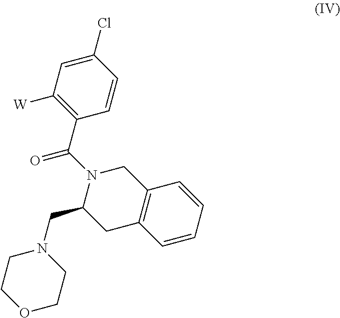

wherein W represents a leaving group selected from halogen atom, trifluoromethanesulfonate group, methanesulfonate group and para-toluenesulfonate group,

In some embodiments, the compound of formula (IV) is synthetized using a 4-chlorobenzoic acid derivative (compound of formula (II)) and (3S)-3-[(morpholin-4-yl)methyl]-1,2,3,4-tetrahydroisoquinoline (compound of formula (I)) as starting materials.

In another embodiment, the compound of formula (V) is further hydrolysed to prepare the carboxylic acid of formula (VI):

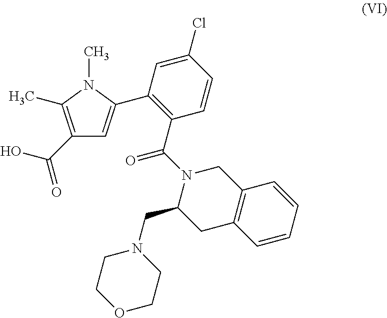 [SMILES: Cc1c(C(=O)O)cc(-c2cc(Cl)ccc2C(=O)N2Cc3ccccc3C[C@H]2CN2CCOCC2)n1C]

In some embodiments, the present invention relates to a process for preparing N-[4-(benzyloxy)phenyl]-5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-1,2-dimethyl-1H-pyrrole-3-carboxamide of formula (VII):

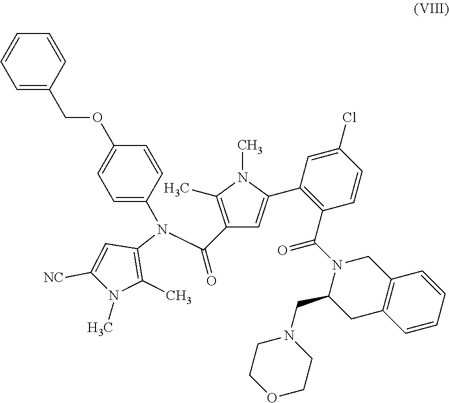 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

using the compound (VI) as defined supra and the compound of formula (VII) as starting materials:

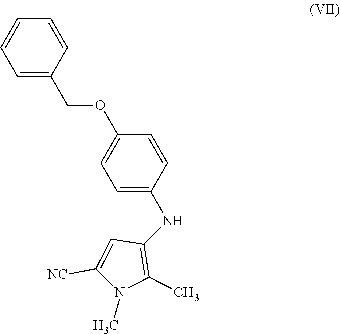 [SMILES: Cc1c(Nc2ccc(OCc3ccccc3)cc2)cc(C#N)n1C]

The compounds of formulae (IV), (V), (VI), (VII) and (VIII) obtained according to the process of the invention are useful in the synthesis of Compound A as well as its structurally-close analogues.

In particular, Compound A has pro-apoptotic properties, notably, it is able to inhibit the anti-apoptotic Bcl-2 protein, which is overexpressed in various types of cancer, making it possible to use Compound A in pathologies involving a defect in apoptosis, such as, for example, in the treatment of cancer and of immune and auto-immune diseases.

In view of the pharmaceutical value of Compound A, it is important to be able to obtain it by an effective synthesis process that is readily transferable to the industrial scale and that results in Compound A in a good yield and with excellent purity, starting from economical and readily obtainable starting materials.

In another aspect, the present invention relates to a process for preparing 4-[4-(benzyloxy)anilino]-1,5-dimethyl-1H-pyrrole-2-carbonitrile of formula (VII) and its application for the production of compound of formula (VIII).

The structure of Compound A is:

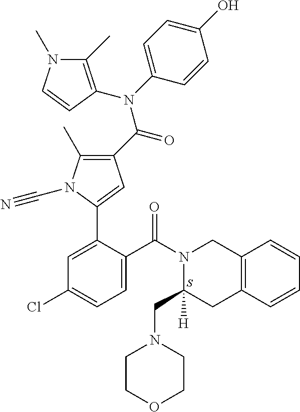

5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide. The preparation of Compound A and its structurally-close analogues, their use as Bcl-2 inhibitors for the treatment of cancer and pharmaceutical formulations thereof, is described in WO 2015/011400, the content of which is incorporated by reference. The preparation is specifically disclosed in Example 386 of WO 2015/011400 in the form of a hydrochloride salt and hydrogen sulfate salt of the same is also described in WO 2020/089281. Furthermore, cyclodextrin-based formulations comprising Compound A are shown in WO 2020/089286.

Particularly, the process for synthesizing Compound A as disclosed in WO 2015/011400 comprised the following steps that are summarized in Scheme 1 below:
(a) a C—H activation of the ethyl 1,2-dimethyl-1H-pyrrole-3-carboxylate with 2-bromo-4-chlorobenzaldehyde;
(b) an oxydation step;
(c) a peptidic coupling;
(d) a saponification step;
(e) a N-acylation step with a secondary amine;
(f) a deprotection step.

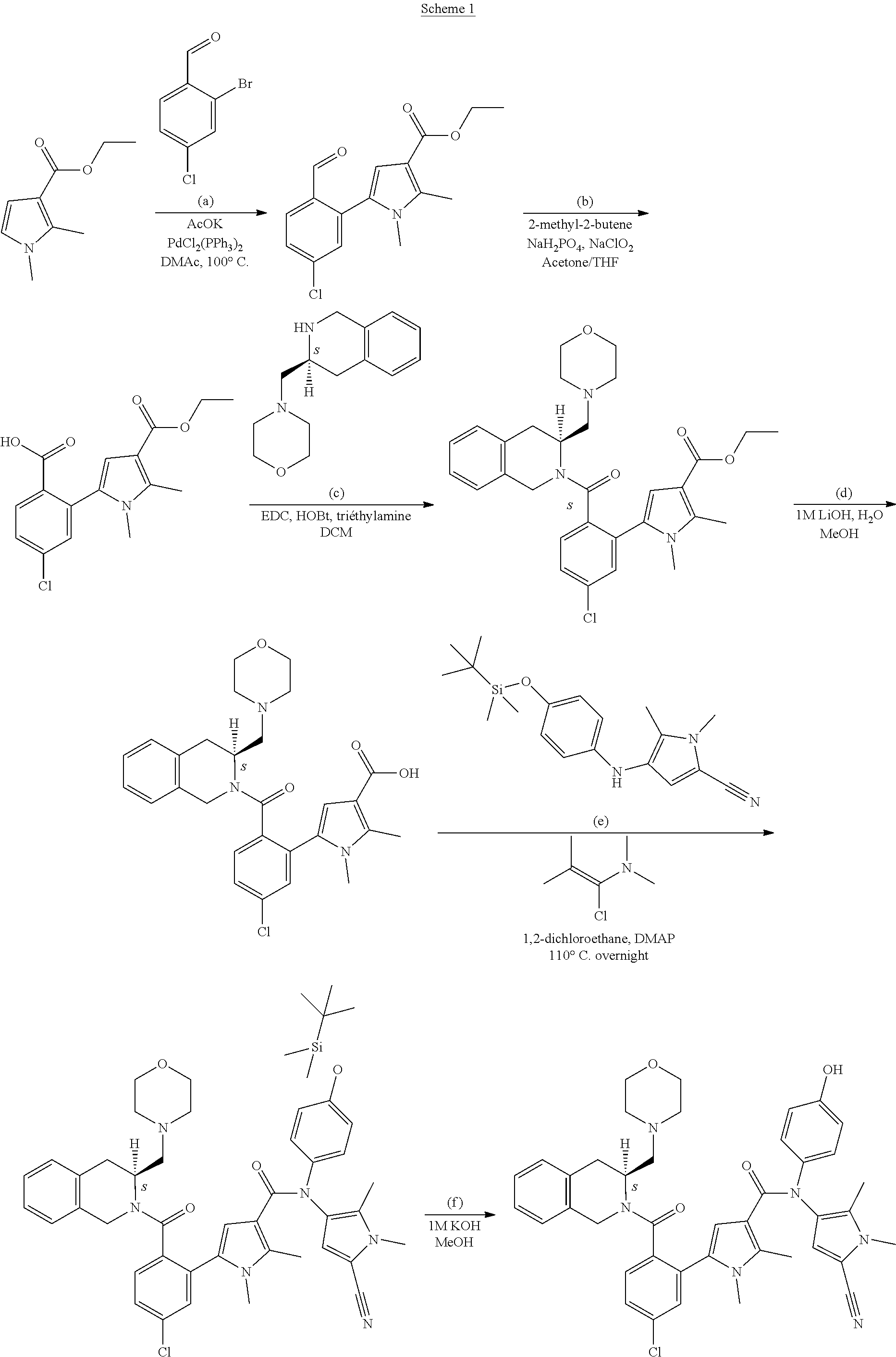

Compound A is obtained in 6 steps using (3S)-3-[(morpholin-4-yl)methyl]-1,2,3,4-tetrahydroisoquinoline, 2-bromo-4-chlorobenzaldehyde, ethyl 1,2-dimethyl-1H-pyrrole-3-carboxylate and 4-({4-[(tert-butyldimethylsilyl)oxy]phenyl }amino)-1,5-dimethyl-1H-pyrrole-2-carbonitrile as starting materials. When transferred to the industrial scale, difficulties in implementing that process rapidly came to light: particularly, the risk of using potential explosive reagent such as hydroxybenzotriazole (HOBt) during the peptidic coupling between (3S)-3-[(morpholin-4-yl)methyl]-1,2,3,4-tetrahydroisoquinoline and 4-chloro-2-[4-(ethoxycarbonyl)-1,5-dimethyl-1H-pyrrol-2-yl]benzoic acid, the risk of using toxic solvent such as N,N-dimethylacetamide (DMAc), and possibly carcinogenic solvent such as 1,2-dichloroethane. In addition, the coupling step (e) with 4-({4-[(tert-butyldimethylsilyl)oxy]phenyl }amino)-1,5-dimethyl-1H-pyrrole-2-carbonitrile requires a long contact time at high temperature and generates some by-products (such as anhydride derivatives) as represented below:

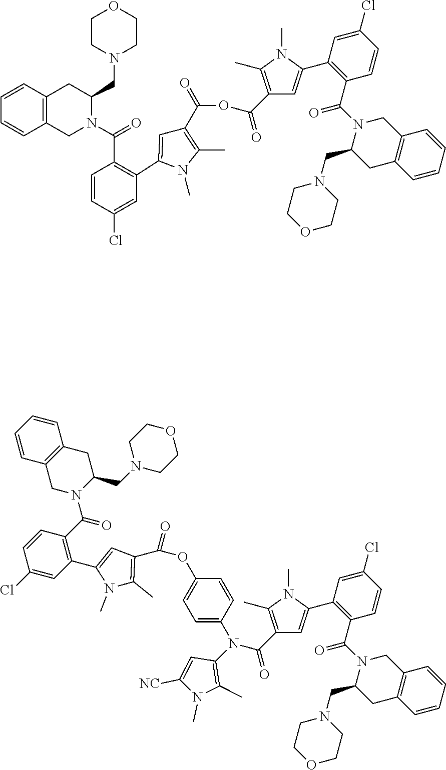

Effort to limit their formation is needed. Furthermore, a substantial variability in yields was observed for step (e) suggesting that the experimental conditions for this coupling step as described WO 2015/011400 are not robust enough for industrial applications. Last, the use of the Ghosez reagent (1-chloro-N,N,2-trimethyl-prop-1-en-1-amine) at industrial scale may be complex due to some stability issue.

Consequently, the search for new efficient synthesis routes is still ongoing and the Applicant has continued his investigations to develop a new synthesis of Compound A, intended to produce large-scale batches. This synthesis yields compounds of formulae (IV), (V), (VI), (VII) and (VIII) in reproducible manner, with excellent yields and with a purity which is compatible with its use as a pharmaceutically acceptable intermediate. In the end, this new process makes it possible to obtain Compound A with a good yield (32% based on the chemical pathway detailed in Scheme 2 below) and with a purity that is compatible with its use as a pharmaceutical active ingredient (superior to 98%, preferably superior to 99%).

More especially, the Applicant has now developed a new synthesis process making it possible to obtain the compounds of formulae (IV), (V) and (VI) in reproducible manner without the need for laborious purification. Similarly to the synthesis process disclosed in WO 2015/011400, (3S)-3-[(morpholin-4-yl)methyl]-1,2,3,4-tetrahydroisoquinoline and ethyl 1,2-dimethyl-1H-pyrrole-3-carboxylate are used as starting materials. However, a 4-chlorobenzoic acid derivative formula (II) is used as a new starting material:

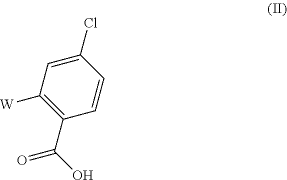

In a preferred embodiment, the compound of formula (II) is 2-bromo-4-chlorobenzoic acid.

This new starting material has the advantage of being simple and readily obtainable in large amounts at less cost. Thus, the process according to the invention is based on a new chemical pathway involving a compound of formula (IV) as an intermediate. More globally, it allows the obtention of Compound A in 5 steps, i.e. one step less as compared to the disclosure of WO 2015/011400. Last, the tert-butyldimethylsilyl was replaced with a benzyl group as a protecting group for the hydroxy function of the N-(5-cyano-1,2-dimethyl-1H-pyrrol-3-yl)-N-(4-hydroxyphenyl) moiety. By doing so, the yield of the coupling reaction between the secondary protected amine and the compound of formula (VII) is higher and reproducible on large scale batches (less variability is observed thanks to robust experimental conditions). Advantageously, this new coupling reaction also avoids the formation of the anhydride derivatives impurities that are discussed supra. The purity of the compound of formula (VIII) so obtained is more easily controlled.

A summary of the synthesis process according to the invention is showed in Scheme 2 below.

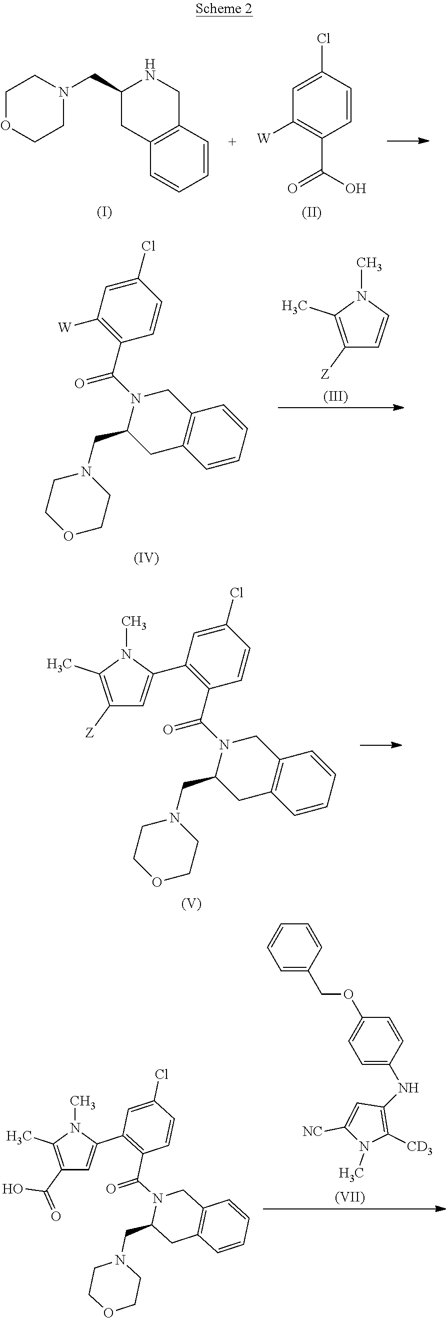

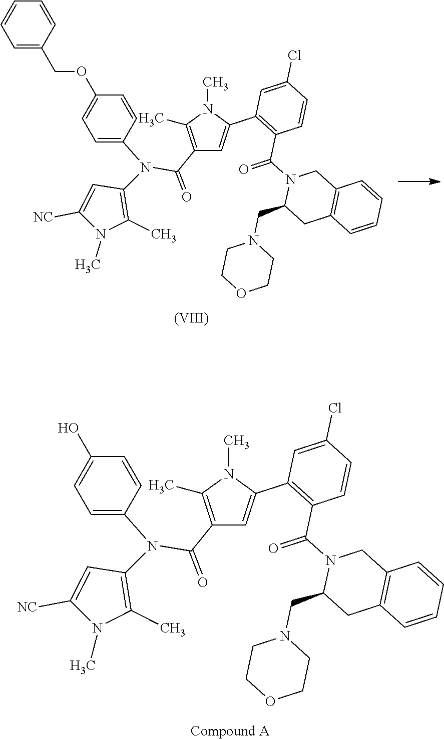 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C ; "(VIII)" ; Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C ; "Compound A"]

### DETAILED DESCRIPTION OF THE INVENTION

In a first embodiment (E1), the present invention provides a process for preparing a compound of formula (V):

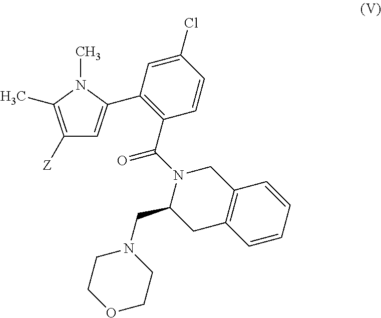

wherein:
Z is a group selected from —COOR and —CN, and
R represents a (C₁-C₆)alkyl group, an allyl group or a —CH₂-aryl group,
comprising the step of reacting a compound of formula (III):

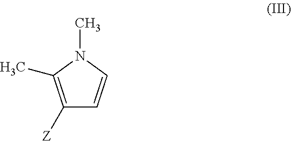

wherein Z is as defined above,
with a compound of formula (IV):

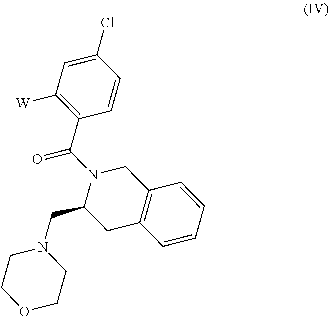

wherein W represents a leaving group selected from halogen atom, trifluoromethanesulfonate group, methanesulfonate group and para-toluenesulfonate group,
in a solvent or a mixture of solvents, at a temperature superior to 70° ° C. in the presence of:
(i) a palladium catalyst;
(ii) optionally a phosphine; and
(iii) a base.

Further enumerated embodiments (E) of the invention are described herein. It will be recognized that features specified in each embodiment may be combined with other specified features to provide further embodiments of the present invention.
E2. A process according to E1, wherein Z is —COOR and R represents a methyl, ethyl, isopropyl, tert-butyl, benzyl or a para-methoxybenzyl group. In a preferred embodiment, R represents an ethyl group.
E3. A process according to E1, wherein W represents a bromine atom.
E4. A process according to E1 to E3, wherein the palladium catalyst is palladium(II)acetate (Pd(OAc)₂).
E5. A process according to E1 to E3, wherein the reaction mixture further contains a phosphine selected from tri-tert-butylphosphine, XPhos, CyJohnPhos and Tri(o-tolyl)-phosphine, preferably CyJohnPhos.
E6. A process according to El to E3, wherein the solvent is an aprotic solvent.
E7.A process according to E6, wherein the solvent is selected from dimethylsulfoxide (DMSO), N-butylpyrrolidinone (NBP), 2-methyltetrahydrofuran and toluene, preferably dimethylsulfoxide.
E8. A process according to E1 to E3, wherein the temperature is superior to 90° C. preferably T=100° C.
E9. A process according to E1 to E3, wherein the base is a carbonate salt, preferably Na₂CO₃,Cs₂CO₃ or K₂CO₃, even more preferably, K2CO3.
E10. A process according to E1 to E₃, wherein the reaction mixture further contains pivalic acid.

In the process according to the first embodiment, the reaction between the compounds of formula (III) and (IV) can be carried out using other catalyst systems than palladium among which there may be mentioned:
rongalite (J. Org. Chem. 2019, 84, 9946-9956);
cadmium sulfide and zinc selenide (photoredox catalysis as described in Chemistry of Materials (2017), 29(12), 5225-5231);
copper/nickel catalyst (photoredox catalysis as described in Organic Letters (2017), 19(13), 3576-3579);
nickel catalyst (Negishi coupling as described in Tetrahedron (2006), 62(32), 7521-7533);
copper/palladium catalyst (Organic Letters (2004), 6(20), 3649-3652);
lithium/nickel catalyst (ChemSusChem (2017), 10(10), 2242-2248);
nickel or iron catalyst (Kumada reaction as described in) with the proviso that this mechanism requires an additional step in order to functionalize the pyrrole group (iodination).
E11. A process according to any one of El to E10, wherein the compound of formula (IV), or an addition salt thereof with a pharmaceutically acceptable acid:

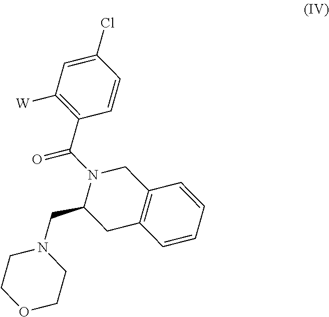

wherein W represents selected from halogen atom, trifluoromethanesulfonate group, methanesulfonate group and para-toluenesulfonate group,
is obtained by a coupling reaction of the compound of formula (I):

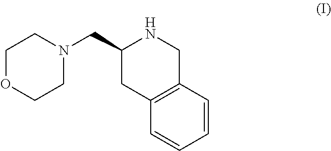 [SMILES: c1ccc2c(c1)CN[C@H](CN1CCOCC1)C2]

or an addition salt thereof with a pharmaceutically acceptable acid,
with a compound of formula (II):

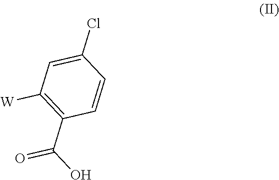

in an aprotic solvent in the presence of an amine base and a coupling agent.
E12. The process according to E11, wherein the compound of formula (II) is 2-bromo-4-chlorobenzoic acid thus leading to the formation of the following compound of formula (IV-a):

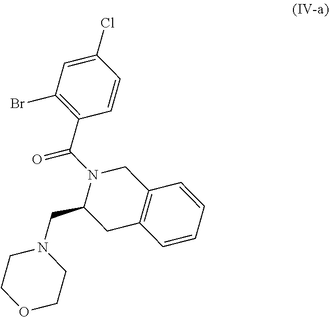 [SMILES: O=C(c1ccc(Cl)cc1Br)N1Cc2ccccc2C[C@H]1CN1CCOCC1]

E13. The process according to E11 or E12, wherein the compound (I) is in the form of a dihydrochloride salt.
E14. The process according to E11 or E12, wherein the coupling agent is selected from propylphosphonic anhydride, cyanuric chloride, methyl propiolate, tetraethyl orthosilicate, pivalyl chloride, N-ethoxycarbonyl-2-ethoxy-1,2-dihydroquinoline, isobutylchloroformate, thionyl chloride and oxalyl chloride, preferably propylphosphonic anhydride.
E15. The process according to E11 or E12, wherein the amine base is selected from triethylamine, N,N-diisopropylethylamine, 1,4-diazabicyclo[2.2.2]octane, 1,8-diazabicyclo[5.4.0]undec-7-ene, N-methylmorpholine, N-ethylmorpholine, pyridine and 2,6-lutidine. In a preferred embodiment, the amine base is triethylamine.
E16. The process according to E11 or E12, wherein the temperature is comprised between 20 to 50° C.
E17. The process according to E11 or E12, wherein the aprotic solvent is selected from ethyl acetate, methylene chloride and isopropyl ether, preferably ethyl acetate.
E18. The process according to E11 or E12, wherein the compound of formula (IV) is isolated as a free base.
E19. The process according to E11 or E12, wherein the compound of formula (IV) is isolated in the form of an addition salt with a pharmaceutically acceptable acid selected from oxalic acid, methanesulfonic acid and hydrochloric acid.
E20. A process according to any one of E1 to E19, wherein the ester or nitrile function of the compound of formula (V):

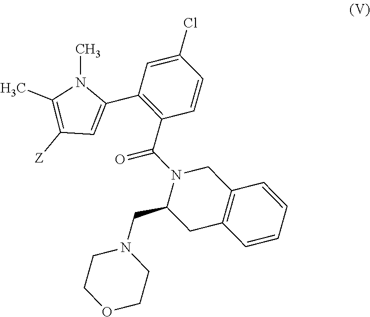

wherein:
Z is a group selected from —COOR and —CN, and
R represents a (C₁-C₆)alkyl group, an allyl group or a —CH₂-aryl group,
is further hydrolysed in a protic medium to yield the compound of formula (VI):

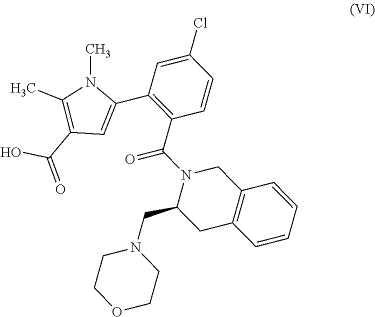 [SMILES: Cc1c(C(=O)O)cc(-c2cc(Cl)ccc2C(=O)N2Cc3ccccc3C[C@H]2CN2CCOCC2)n1C]

which compound of formula (VI) is further isolated as a zwitterion or in the form of an addition salt thereof with a pharmaceutically acceptable acid, before being subjected to a peptidic coupling with 4-[4-(benzyloxy)anilino]-1,5-dimethyl-1H-pyrrole-2-carbonitrile of formula (VII):

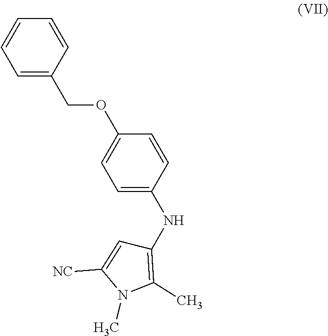 [SMILES: Cc1c(Nc2ccc(OCc3ccccc3)cc2)cc(C#N)n1C]

in an aprotic solvent in the presence of a coupling agent and optionally in the presence of an amine base,
to yield the compound of formula (VIII):

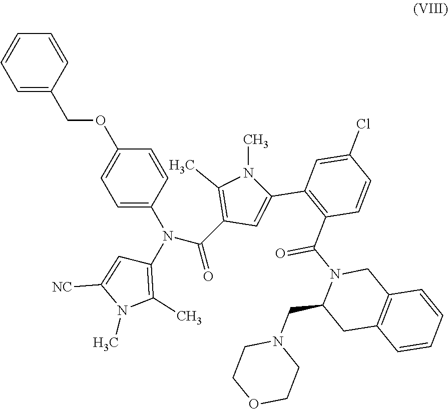 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

which compound of formula (VIII) is deprotected under acidic conditions to yield the Compound A:

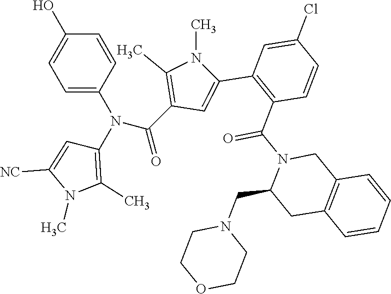 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

which compound is isolated and may be further converted into its addition salts with a pharmaceutically acceptable acid or base.
E21. The process according to E20, wherein Z is —COOR and R represents a methyl, ethyl, isopropyl, tert-butyl, benzyl or a para-methoxybenzyl group.
E22. The process according to E20 wherein the compound of formula (V) is:

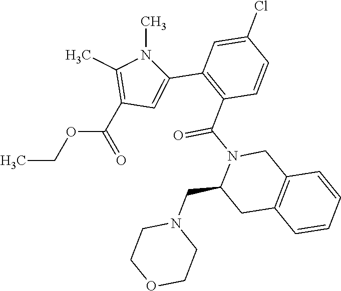 [SMILES: CCOC(=O)c1cc(-c2cc(Cl)ccc2C(=O)N2Cc3ccccc3C[C@H]2CN2CCOCC2)n(C)c1C]

which compound is further hydrolysed under basic conditions.
E23. The process according to E20 wherein the compound of formula (V) is:

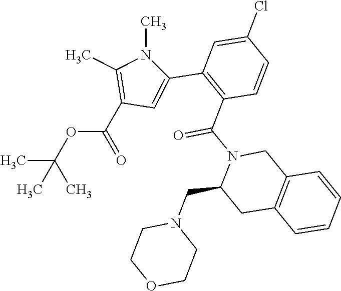 [SMILES: Cc1c(C(=O)OC(C)(C)C)cc(-c2cc(Cl)ccc2C(=O)N2Cc3ccccc3C[C@H]2CN2CCOCC2)n1C]

which compound is further hydrolysed under acidic conditions.
E24. The process according to E20, wherein the protic medium used for the hydrolysis of compound (V) is methanol, ethanol, isopropanol, DMSO/water or an ethanol/water mixture, preferably ethanol.
E25. The process according to E24, wherein the protic medium used is ethanol/water and the hydrolysis of compound (V) is performed at a temperature comprised between 60 and 80° C.
E26. The process according to E20 wherein the compound of formula (VI) is isolated in the form of an addition salt with a pharmaceutically acceptable acid selected from hydrochloric acid, sulfuric acid, hydrobromic acid, para-toluenesulfonic acid, methanesulfonic acid, 1,5-naphtalenedisulfonic, acetic acid, trifluoroacetic acid, fumaric acid, tartric acid, oxalic acid, citric acid, succinic acid, maleic acid, phosphoric acid and boric acid. In a preferred embodiment, the pharmaceutically acceptable acid is selected from hydrochloric acid, sulfuric acid, hydrobromic acid, para-toluenesulfonic acid, methanesulfonic acid, 1,5-naphtalenedisulfonic, phosphoric acid and boric acid. Even more preferably, the compound of formula (VI) is isolated in the form of a hydrochloric acid salt.
E27. The process according to E20 wherein the coupling agent is selected from thionyl chloride, isobutyl chloroformate, N-ethoxycarbonyl-2-ethoxy-1,2-dihydroquinoline and propylphosphonic anhydride. In a preferred embodiment, the coupling agent is propylphosphonic anhydride.
E28. The process according to E20 wherein the aprotic solvent used for the peptidic coupling is selected from dichloromethane, acetonitrile, toluene, ethyl acetate, butyl acetate, isobutyl acetate, propyl acetate, isopropyl acetate, chlorobenzene, N,N-dimethylformamide and pyridine. In a preferred embodiment, a high boiling point solvent is used; it is selected from toluene, butyl acetate, isobutyl acetate, propyl acetate, isopropyl acetate, chlorobenzene, N,N-dimethylformamide and pyridine.
E29. The process according to wherein the coupling agent is N-ethoxycarbonyl-2-ethoxy-1,2-dihydroquinoline and the solvent is toluene.
E30. The process according to E20 wherein an amine base is used for the peptidic coupling.
E31. The process according to E30 wherein the amine base used for the peptidic coupling of the compound of formula (VI) with the compound of formula (VII) is selected from pyridine, N,N-diisopropylethylamine and triethylamine. In a preferred embodiment, the amine base is pyridine.
E32. The process according to E20 wherein the coupling agent is propylphosphonic anhydride and the amine base is pyridine.
E33. The process according to E20 wherein the coupling agent is propylphosphonic anhydride, the amine base is pyridine and the aprotic solvent is selected from: acetonitrile, toluene, chlorobenzene, ethyl acetate, butyl acetate and propyle acetate, more preferably the aprotic solvent is selected from toluene, chlorobenzene and butyl acetate, even more preferably the aprotic solvent is chlorobenzene.
E34. The process according to E33 wherein the peptidic coupling is performed at a temperature comprised between 60 and 135° C., preferably between 110° C. and 135° C., even more preferably 120° C.
E35. The process according to E20 wherein the deprotection of the compound of formula (VIII) is performed in the presence of hydrobromic acid, hydrochloric acid, sulfuric acid, methanesulfonic acid, a mixture of hydrochloric acid and acetic acid, or a mixture of hydrobromic acid and acetic acid, more preferably in the presence of a mixture of hydrobromic acid and acetic acid.
E36. The process according to E35 wherein the solvent used for the deprotection of the compound of formula (VIII) is selected from dichloromethane, chlorobenzene, dioxane and ethyl acetate, more preferably ethyl acetate.
E37. The process according to E35 or E36 wherein the temperature is maintained below 40° C.
E38. The process according to E20 wherein the deprotection of the compound of formula (VIII) is performed via a hydrogenation reaction in the presence of a catalyst under acidic conditions.
E39. The process according to E38 wherein:
the palladium catalyst is Pd(OH)₂ on carbon or palladium on carbon,
the hydrogenation reaction is performed in hydrochloride ethanol at a temperature comprised between 40 and 65° ° C., preferably between 45 and 60° ° C.
E40. The process according to any one of E20 to E39, wherein the compound of formula (VII):

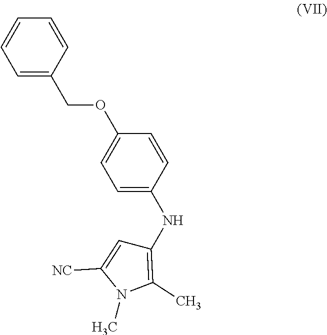 [SMILES: Cc1c(Nc2ccc(OCc3ccccc3)cc2)cc(C#N)n1C]

is obtained by the reaction of the compound of formula (SM1-VIII):

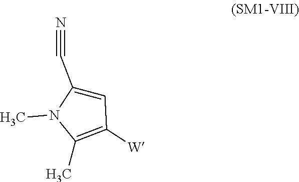

wherein W′ represents a leaving group selected from halogen atom, trifluoromethanesulfonate group, methanesulfonate group and para-toluenesulfonate group,
with the compound of formula (SM2-VIII):

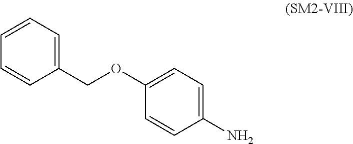 [SMILES: Nc1ccc(OCc2ccccc2)cc1]

in the presence of a palladium-phosphine complex catalyst and a base in a polar aprotic solvent at a temperature comprised between 40 and 85° C.,
wherein the palladium-phosphine complex catalyst is ready for use or prepared in situ starting from a palladium catalyst and a phosphine.
E41. A process for preparing the Compound A:

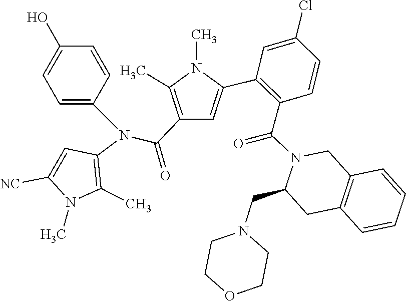 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

characterized in that the compound of formula (I):

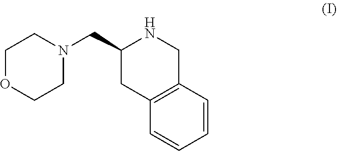 [SMILES: c1ccc2c(c1)CN[C@H](CN1CCOCC1)C2]

or an addition salt thereof with a pharmaceutically acceptable acid, is subjected to a coupling reaction with the compound of formula (II):

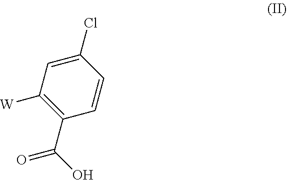

wherein W represents a leaving group selected from halogen atom, trifluoromethanesulfonate group, methanesulfonate group and para-toluenesulfonate group,
in an aprotic solvent in the presence of an amine base and a coupling agent at a temperature comprised between 20 to 50° C.,
to yield the compound of formula (IV):

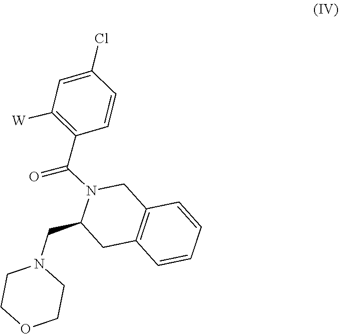

which compound of formula (IV) is reacted with a compound of formula (III):

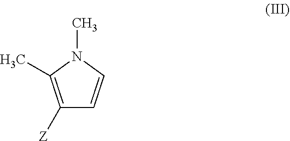

wherein:
Z is a group selected from —COOR and —CN, and
R represents a (C1-C6)alkyl group, an allyl group or a —CH₂-aryl group,
in a solvent or a mixture of solvents, at a temperature superior to 70° C. in the presence of:
   (i) a palladium catalyst;
   (ii) optionally a phosphine; and
   (iii) a base,
to yield the compound of formula (V):

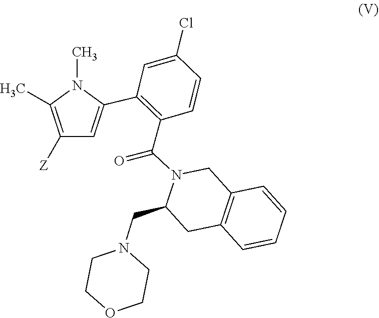

the ester or nitrile function of which compound of formula (V) is further hydrolysed in a protic medium to yield the compound of formula (VI):

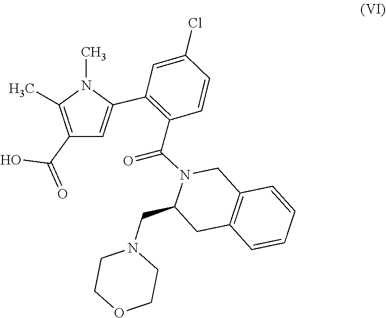 [SMILES: Cc1c(C(=O)O)cc(-c2cc(Cl)ccc2C(=O)N2Cc3ccccc3C[C@H]2CN2CCOCC2)n1C]

which compound of formula (VI) is further isolated as a zwitterion or in the form of an addition salt thereof with a pharmaceutically acceptable acid, before being subjected to a peptidic coupling with 4-[4-(benzyloxy)anilino]-1,5-dimethyl-1H-pyrrole-2-carbonitrile of formula (VII):

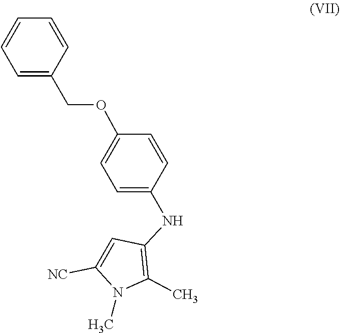 [SMILES: Cc1c(Nc2ccc(OCc3ccccc3)cc2)cc(C#N)n1C]

in an aprotic solvent in the presence of a coupling agent and optionally in the presence of an amine base,
to yield the compound of formula (VIII):

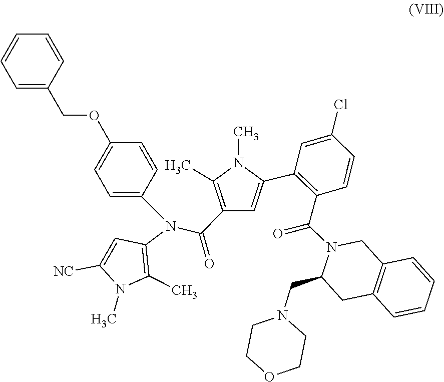 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

which compound of formula (VIII) is deprotected under acidic conditions to yield the Compound A:

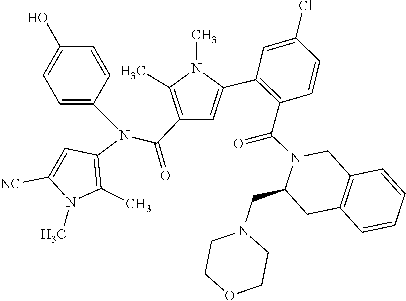 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

which compound is isolated and may be further converted into its addition salts with a pharmaceutically acceptable acid or base.
E42. The process according to E41, wherein Compound A is isolated in solution.
E43. The process according to E41, wherein the compound of formula (VII):

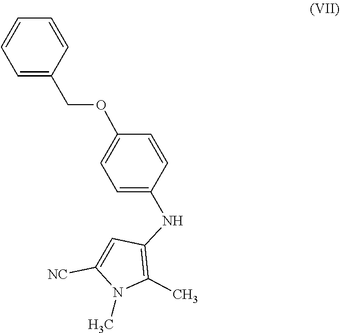 [SMILES: Cc1c(Nc2ccc(OCc3ccccc3)cc2)cc(C#N)n1C]

is obtained by the reaction of the compound of formula (SM1-VIII):

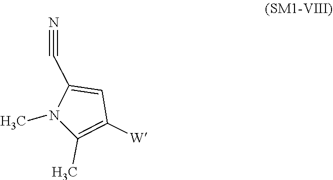

wherein W′ represents a leaving group selected from halogen atom, trifluoromethanesulfonate group, methanesulfonate group and para-toluenesulfonate group,
with the compound of formula (SM2-VIII):

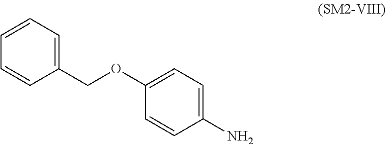 [SMILES: Nc1ccc(OCc2ccccc2)cc1]

in the presence of a palladium-phosphine complex catalyst and a base in a polar aprotic solvent at a temperature comprised between 40 and 85° C.,
wherein the palladium-phosphine complex catalyst is ready for use or prepared in situ starting from a palladium catalyst and a phosphine.
E44. Compound of formula (IV), or an addition salt thereof with a pharmaceutically acceptable acid:

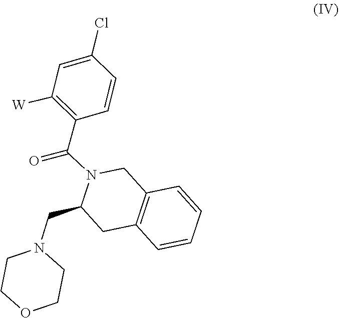

wherein W represents a leaving group selected from halogen atom, trifluoromethanesulfonate group, methanesulfonate group and para-toluenesulfonate group.
E45. Compound of formula (V):

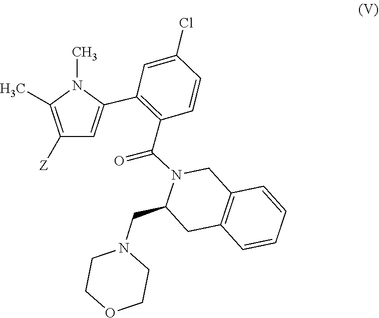

wherein:
Z is a group selected from —COOR and —CN, and
R represents a (C₁-C₆)alkyl group, an allyl group or a —CH₂-aryl group, with the proviso that the (C₁-C₆)alkyl group does not represent an ethyl group.
E46. Compound of formula (VII):

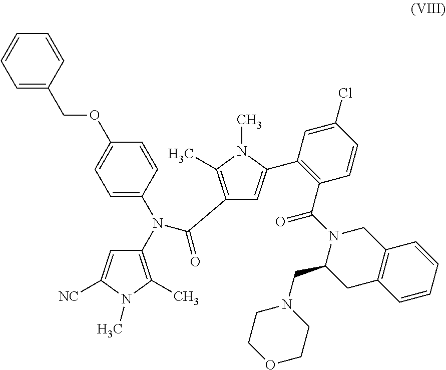 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

E47. Compound of formula (VII):

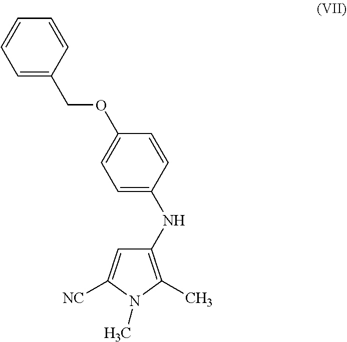 [SMILES: Cc1c(Nc2ccc(OCc3ccccc3)cc2)cc(C#N)n1C]

E48. A process for the preparation of the compound of formula (VII) comprising the step of reacting a compound of formula (SM1-VIII):

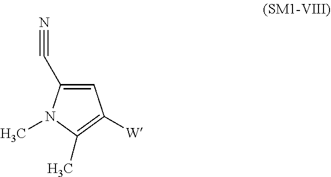

wherein W′ represents a leaving group selected from halogen atom, trifluoromethanesulfonate group, methanesulfonate group and para-toluenesulfonate group,
with the compound of formula (SM2-VIII):

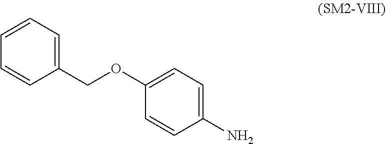 [SMILES: Nc1ccc(OCc2ccccc2)cc1]

in the presence of a palladium-phosphine complex catalyst and a base in a polar aprotic solvent at a temperature comprised between 40 and 85° C.,
wherein the palladium-phosphine complex catalyst is ready for use or prepared in situ starting from a palladium catalyst and a phosphine.
E49. The process according to E48 wherein W′ represents a bromine atom.
E50. The process according to E48 or E49 wherein the solvent is selected from N,N-dimethylformamide, dimethylsulfoxide and 2-methyltetrahydrofuran, more preferably 2-methyltetrahydrofuran.
E51. The process according to E48 or E49 wherein the palladium-phosphine complex catalyst is selected from tBuXPhos Pd G1, tBuXPhos Pd G3, BrettPhos G3, tBuXPhosPd(allyl)OTf, more preferably tBuXPhosPd(allyl)OTf.
E52. The process according to E48 or E49 wherein the palladium-phosphine complex catalyst is prepared in situ starting from Pd₂dba₃ and tBuXPhos.
E53. The process according to E48 or E49 wherein the base is selected from fBuONa, tBuOK, K₃PO₄ and K₂CO₃, more preferably tBuONa.

A particular embodiment of the present invention relates to a process for the preparation of the compound of formula (IV), or an addition salt thereof with a pharmaceutically acceptable acid:

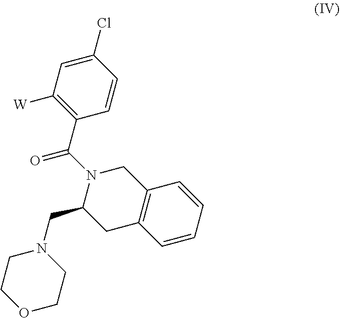

wherein W represents a leaving group selected from halogen atom, trifluoromethanesulfonate group, methanesulfonate group and para-toluenesulfonate group,
which is obtained by a coupling reaction of the compound of formula (I):

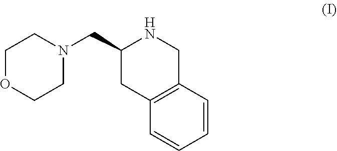 [SMILES: c1ccc2c(c1)CN[C@H](CN1CCOCC1)C2]

or an addition salt thereof with a pharmaceutically acceptable acid, with a compound of formula (II):

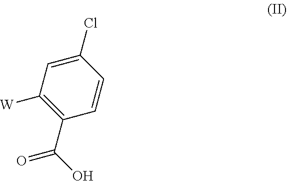

in an aprotic solvent in the presence of an amine base and a coupling agent at a temperature comprised between 20 to 50° C.

Specific embodiments of the preparation of the compound of formula (IV) are detailed in E12 to E19 and apply to this independent process step.

The present invention also relates to the preparation of the compound of formula (VIII):

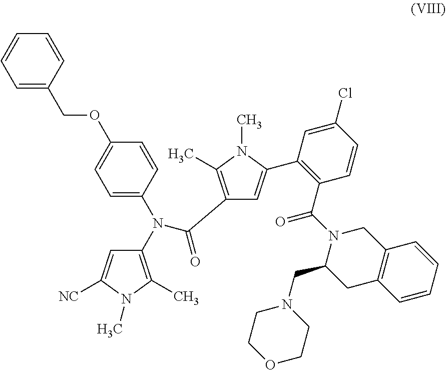 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

which is obtained by a peptidic coupling between the compound of formula (VI):

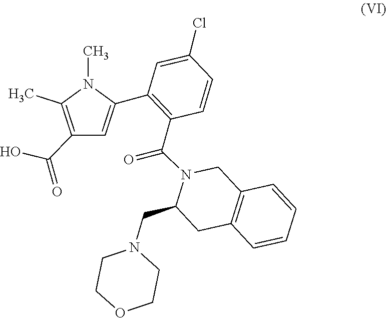 [SMILES: Cc1c(C(=O)O)cc(-c2cc(Cl)ccc2C(=O)N2Cc3ccccc3C[C@H]2CN2CCOCC2)n1C]

with 4-[4-(benzyloxy)anilino]-1,5-dimethyl-1H-pyrrole-2-carbonitrile of formula (VII):

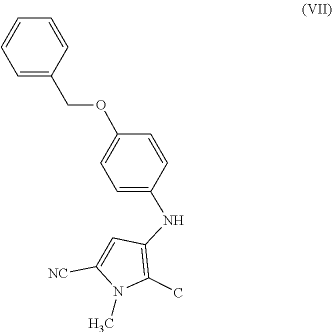

in an aprotic solvent in the presence of a coupling agent and optionally in the presence of an amine base.

Specific embodiments of the preparation of the compound of formula (VIII) are detailed in E27 to E34 and apply to this independent process step.

The present process is especially advantageous for the following reasons:
it makes it possible to obtain the Compound A on the industrial scale in a reproducible manner and in excellent yields starting from a simple and low-cost starting material;
it makes it possible to obtain the compounds of formulae (IV), (V), (VI) and (VII) on the industrial scale in a reproducible manner and in excellent yields starting from a simple and low-cost starting material without the need for laborious purification;
it makes it possible to avoid the use of highly inflammable and toxic reagents;
it makes it possible to achieve high levels of purity using standard crystallization techniques.

The present invention also relates to the use of the compound of formula (IV) for the synthesis of Compound A.

The present invention also relates to the use of the compounds of formulae (VII) and (VIII) for the synthesis of Compound A.

In another embodiment, the present invention relates to the use of some compounds of formula (V) as defined hereinafter for the synthesis of Compound A:

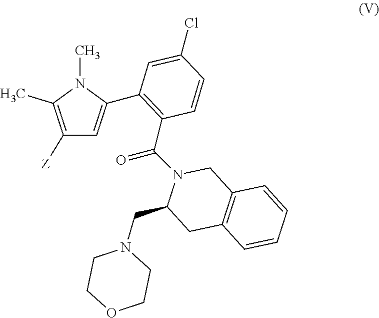

wherein:
Z is a group selected from —COOR and —CN, and
R represents a (C1-C6)alkyl group, an allyl group or a —CH₂-aryl group, with the proviso that the (C₁-C₆)alkyl group does not represent an ethyl group.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “aryl” as used herein, refers to a phenyl optionally substituted by a methoxy group, naphthyl, biphenyl or indenyl group.

The term “halogen atom” as used herein refers preferably to iodine, bromine and chlorine.

The term “medium” means the phase (and composition of the phase) in which the chemical reactions are carried out. As used herein, it refers to a solvent or a mixture of solvents.

Some abbreviations are defined below:
tBuONa: sodium tert-butoxide
tBuOK: potassium tert-butoxide
tBuXPhos Pd G3: [(2-di-tert-butylphosphino-2′,4′,6′-triisopropyl-1,1′-biphenyl)-2-(2′′-amino-1,1′-biphenyl)] palladium(II) methanesulfonate
tBuXPhos Pd G1: [2-(di-tert-butylphosphino)-2′,4′, 6′-triisopropyl-1, 1′-biphenyl][2-(2-aminoethyl)phenyl)]palladium(II) chloride
BrettPhos Pd G3: [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate
tBuXPhosPd(allyl)OTf: allyl(2-di-tert-butylphosphino-2′,4′,6′-triisopropyl-1,1′-biphenyl) palladium(II) triflate
DCM: dichloromethane or methylene chloride
DMAc: N,N-dimethylacetamide
DMAP: 4-dimethylaminopyridine
EDC: 1-ethyl-3-(3′-dimethylaminopropyl)-carbodiimide
HOBt: hydroxybenzotriazole
XPhos: 2-dicyclohexylphosphino-2′,4′, 6′-triisopropylbiphenyl
CyJohnPhos: 2-(dicyclohexylphosphino)biphenyl
Pd₂dba₃: tris(dibenzylideneacetone)dipalladium(0)
THF: tetrahydrofuran

Preferably, the reactants are agitated during the reaction period using suitable mechanical agitators or stirrers. The reactions can be conducted from about 2 to about 24 hours or more, depending on the temperatures, dilution volumes, catalysts, concentrations and/or nature of the materials in the reaction mixtures. The term ‘about’ as used herein means +/−5%, in particular +/−2%, more particularly +/−1%.

The structures of the compounds described were confirmed by the usual spectroscopic techniques. For example, ¹H NMR data is in the form of delta values, given in part per million (ppm), using the residual peak of the solvent (7.24 ppm for CDCl₃ or 2.49 ppm for DMSOd6 or 33.1 ppm for CD₃OD) as internal standard. Splitting patterns are designated as: s (singlet), d (doublet), t (triplet), m (multiplet), br or brs (broad singlet).

The Preparations herein below illustrate the invention but do not limit it in any way.

### STEP A1—Preparation of 4-bromo-1,5-dimethyl-1H-pyrrole-2-carbonitrile

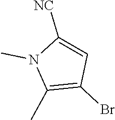

1,5-Dimethyl-1H-pyrrole-2-carbonitrile (1.00 kg) is dissolved in acetonitrile (3.13 kg) and then cooled to 0±5° C. A solution of N-bromosuccinimide (1.52 kg) in acetonitrile (8.86 kg) is added in the course of 2-3 hours while the temperature is maintained at 0±5° C. Once the conversion is complete, the reaction mixture is transferred to cold water. The product is filtered and then washed twice with water. After drying at 40° C., 4-bromo-1,5-dimethyl-1H-pyrrole-2-carbonitrile is isolated in the form of a beige powder with a yield of 92% (Purity by HPLC≥99.0%).

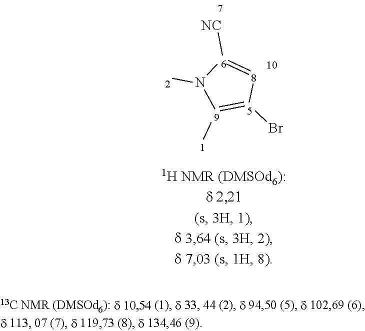

### STEP A2—Preparation of 4-4-(benzyloxy)anilinol-1,5-dimethyl-1H-pyrrole-2-carbonitrile

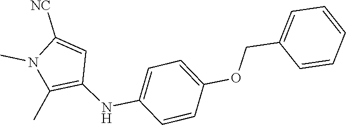 [SMILES: Cc1c(Nc2ccc(OCc3ccccc3)cc2)cc(C#N)n1C]

### Method 1

4-(Benzyloxy)aniline, HCl (1.00 kg) and sodium tert-butoxide (1.22 kg) are suspended in 2-methyltetrahydrofuran (6.00 kg) at 20° C. before being heated to 60° C. After one hour's contact, tBuXPhosPd(allyl)OTf (0.15 kg) is added, followed by a solution of 4-bromo-1,5-dimethyl-1H-pyrrole-2-carbonitrile (0.84 kg) in 2-methyltetrahydrofuran (3.20 kg) in the course of approximately one hour. After 30 minutes' contact, the reaction mixture is cooled to 20° C. IN HCl solution is added until a pH of 3.0±0.5 is obtained. The aqueous phase is removed and then the organic phase is washed twice with a solution of N-acetyl-L-cysteine in water and then again with 1N HCl solution. The organic phase is subjected to a volume reduction in vacuo and then, isobutanol is added at 20° C. The product precipitates during this addition. The suspension is cooled to 5° C. and then filtered. The cake is washed with isobutanol and then heptane before being dried in an oven in vacuo at 40° C. 4-[4-(Benzyloxy)anilino]-1,5-dimethyl-1H-pyrrole-2-carbonitrile is isolated in the form of a white powder with a yield of 70% (Purity by HPLC≥98.0%).

### Method 2

Sodium tert-butoxide (1.22 kg), 4-(benzyloxy)aniline, HCl (1.00 kg), tris(dibenzylideneacetone)dipalladium(0) (97.1 g) and tBuXPhos (90.8 g) are suspended under argon in 2-methyltetrahydrofuran (6.34 kg) at 20° C. before being heated to 40° C. After one hour of contact, a solution of 4-bromo-1,5-dimethyl-1H-pyrrole-2-carbonitrile (0.836 kg) in 2-methyltetrahydrofuran (2.72 kg) is added in the course of approximately one hour, not exceeding 55° C. temperature. After 30 minutes of contact, the reaction mixture is cooled to 20° C. 1N HCl solution is added until a pH of 2.0±0.5. The aqueous phase is removed and then the organic phase is washed twice with 7.5 w% solution of N-acetyl-L-cysteine in water and then again with 1N HCl solution. The organic phase is subjected to a volume reduction in vacuo and then, isobutanol is added at 50° C. The product precipitates during evaporation. The suspension is cooled to 0-5° C. and filtered. The cake is washed with isobutanol and heptane before being dried in an oven in vacuo at 40° C. 4-[4-(Benzyloxy)anilino]-1,5-dimethyl-1H-pyrrole-2-carbonitrile is isolated in the form of yellow powder with a yield of 85% (Purity by HPLC≥98.0%).

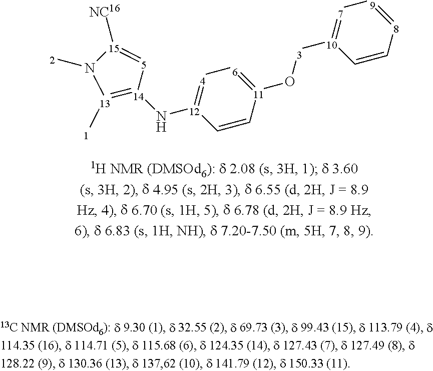

### STEP 1—Preparation of (2-bromo-4-chlorophenyl)[(3S)-3-[(morpholin-4-yl)methyl]-3,4-dihydroisoquinolin-2(1H)-yl]methanone

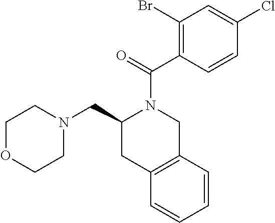 [SMILES: O=C(c1ccc(Cl)cc1Br)N1Cc2ccccc2C[C@H]1CN1CCOCC1]

2-Bromo-4-chlorobenzoic acid (1.000 kg) and (3S)-3-[(morpholin-4-yl)methyl]-1,2,3,4-tetrahydroisoquinoline, 2HCl (1.296 kg) are suspended in ethyl acetate (7.216 kg) at 35° C. Triethylamine (2.148 kg) is then added while the temperature is maintained at 35° C. 50% propylphosphonic anhydride in ethyl acetate (4.595 kg) is added to the reaction mixture in the course of 2.5 hours and contact is then maintained for an additional 1.5 hours at 35° C. The reaction mixture is hydrolysed by the addition of water and sodium hydroxide at 35° C. until a pH of 7.0±0.2 is reached. The two-phase mixture is cooled to 20° C. and then the aqueous phase is removed. The organic phase is washed twice with water and then concentrated until all the residual triethylamine has been removed. The solution is cooled to 20° C. and then isopropyl ether is added (1.095 kg). Once crystallised, the suspension is cooled to 0° C. After a contact time, the product is filtered, washed with isopropyl ether and dried in an oven. (2-Bromo-4-chlorophenyl)[(3S)-3-[(morpholin-4-yl)methyl]-3,4-dihydroisoquinolin-2(1H)-yl]methanone is isolated in the form of a white powder with a yield of 80% (purity by HPLC≥99.0%).

Alternatively, the crystallisation can be initiated via seed addition.

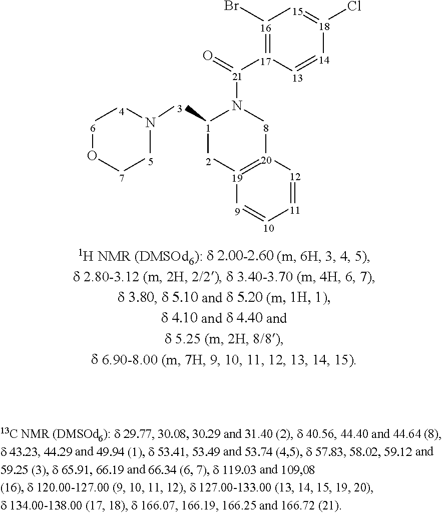

### STEP 2—Preparation of ethyl 5-{5-chloro-2-[(3S)-3-[(morpholin-4-yl)methyl]-3,4-dihydroisoquinoline-2(1H)-carbonyl]phenyl}-1,2-dimethyl-1H-pyrrole-3-carboxylate

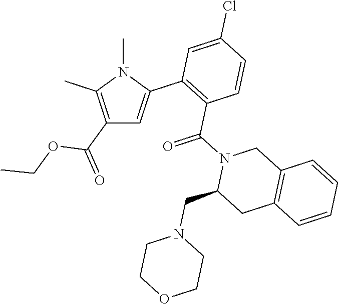 [SMILES: CCOC(=O)c1cc(-c2cc(Cl)ccc2C(=O)N2Cc3ccccc3C[C@H]2CN2CCOCC2)n(C)c1C]

(2-Bromo-4-chlorophenyl)[(3S)-3-[(morpholin-4-yl)methyl]-3,4-dihydroisoquinolin-2(1H)-yl]methanone (1.00 kg), potassium carbonate (0.68 kg), palladium acetate (0.05 kg) and ethyl 1,2-dimethyl-1H-pyrrole-3-carboxylate (0.28 kg) are dissolved in DMSO (5.51 kg) and then the mixture is heated at 100° C. for 24 hours. At the end of the conversion, the reaction mixture is cooled to 50° C., clarified on Clarcel and then rinsed with DMSO and ethyl acetate. The filtrate is cooled to 20° C. and then hydrolysed with water. The product is extracted with ethyl acetate. The organic phase is washed twice with N-acetyl-L-cysteine solution in order to remove the residual palladium and then the pH is adjusted to 8.0±0.2 with aqueous potassium carbonate solution. The aqueous phases are then removed and then the organic phase is washed a final time with water. It is subjected to a volume reduction in vacuo and isopropyl ether is added at 50° C. The suspension is cooled to 5° C. The product is filtered and then the cake is washed with isopropyl ether before being dried in an oven in vacuo. Ethyl 5-{5-chloro-2-[(3S)-3-[(morpholin-4-yl)methyl]-3,4-dihydroisoquinoline-2(1H)-carbonyl]phenyl}-1,2-dimethyl-1H-pyrrole-3- carboxylate is isolated in the form of a brown powder with a yield of approximately 70% (purity by HPLC≥96.0%).

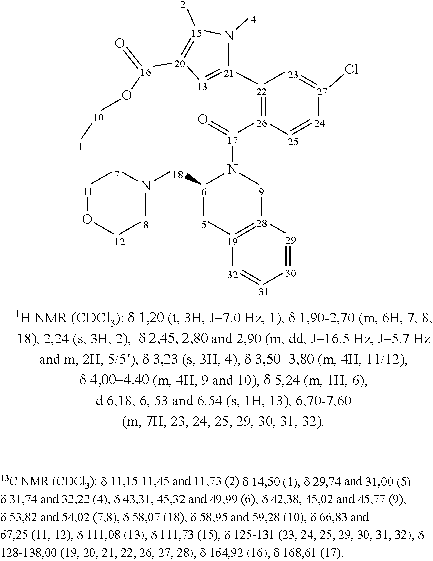

### STEP 3—Preparation of 5-{5-chloro-2-1(3S)-3-[(morpholin-4-yl)methyll-3,4-dihydroisoquinoline-2(1H)-carbonyl]phenyl}-1,2-dimethyl-1H-pyrrole-3-carboxylic acid, hydrochloride

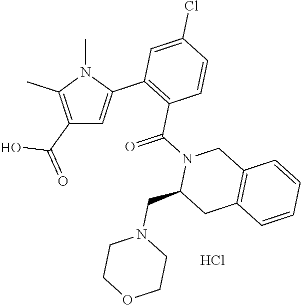 [SMILES: Cc1c(C(=O)O)cc(-c2cc(Cl)ccc2C(=O)N2Cc3ccccc3C[C@H]2CN2CCOCC2)n1C ; Cl]

### Method 1

5-{5-Chloro-2-[(3S)-3-[(morpholin-4-yl)methyl]-3,4-dihydroisoquinoline-2(1H)-carbonyl]phenyl}-1,2-dimethyl-1H-pyrrole-3-carboxylate (1.000 kg) is dissolved in ethanol (4.734 kg) at 20° C. and then 10N sodium hydroxide is added (0.876 kg; 3.5 eq.). The mixture is heated at 75° C. until conversion is complete. After cooling, dilute hydrochloric acid solution is added to pH=1.3. The suspension is cooled to 5° C. and then filtered. The product is washed with water before being dried. 5-{5-Chloro-2-[(3S)-3-[(morpholin-4-yl)methyl]-3,4-dihydroisoquinoline-2(1H)-carbonyl]phenyl }-1,2-dimethyl-1H-pyrrole-3-carboxylic acid, hydrochloride is isolated in the form of a white powder with a yield of 85% (purity by HPLC≥98.0%).

Alternatively, the crystallisation can be initiated via seed addition.

### Method 2.

5-{5-Chloro-2-[(3S)-3-[(morpholin-4-yl)methyl]-3,4-dihydroisoquinoline-2(1H)-carbonyl]phenyl}-1,2-dimethyl-1H-pyrrole-3-carboxylate (1.000 kg) is dissolved in a mixture of ethanol (2.370 Kg) and water (2.000 Kg) at 20° C. and then a 10N sodium hydroxide solution is added (0.876 kg; 3.5 eq.). The mixture is heated and maintained at 80° C. until complete conversion. Ethanol is eliminated by distillation and the volume is adjusted to 5 L with water. At 25° C., this mixture is added to a mixture of isopropanol, water and a concentrated hydrochloric acid solution (0.992 Kg; 5 eq.). After precipitation, the suspension is filtered, washed with water (2×4.000 L/Kg) before being dried. 5-{5-Chloro-2-[(3S)-3-[(morpholin-4-yl)methyl]-3,4-dihydroisoquinoline-2(1H)-carbonyl]phenyl}-1,2-dimethyl-1H-pyrrole-3-carboxylic acid, hydrochloride is isolated with a yield of 97%.

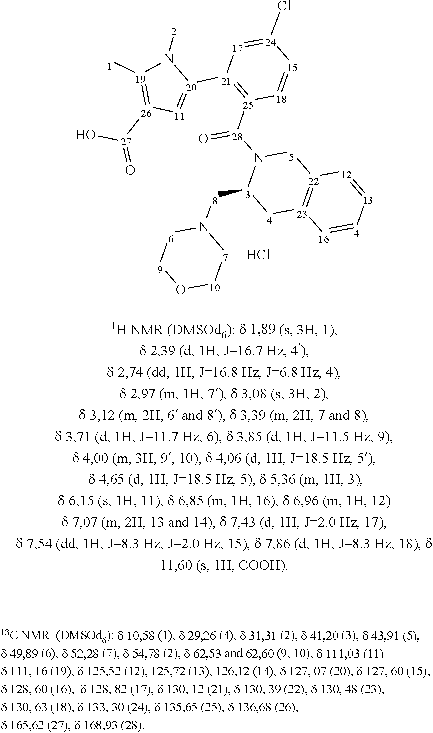

### STEP 4—Preparation of N-J[4-(benzyloxy)phenyl]-5-{5-chloro-2-1(3S)-3-[(morpholin-4-yl)methyl]-3,4-dihydroisoquinoline-2(1H)-carbonyl|phenyl}-N-(5-cyano-1,2-dimethyl-1H-pyrrol-3-yl)-1,2-dimethyl-1H-pyrrole-3-carboxamide

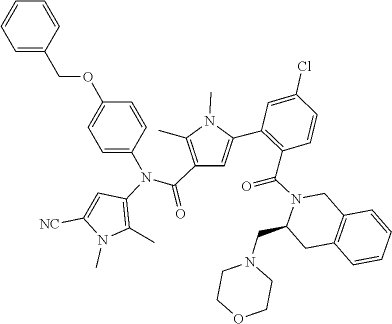 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

### Method 1

5-{5-Chloro-2-[(3S)-3-[(morpholin-4-yl)methyl]-3,4-dihydroisoquinoline-2(1H)-carbonyl]phenyl}-1,2-dimethyl-1H-pyrrole-3-carboxylic acid, HCl (1.000 kg) and 4-[4-(benzyloxy)anilino]-1,5-dimethyl-1H-pyrrole-2-carbonitrile (0.549 kg) are suspended in chlorobenzene (11.10 kg) and then the mixture is heated to 120° C. Pyridine (0.547 kg) as well as 50% propylphosphonic anhydride in ethyl acetate (1.650 kg) are added in succession. After complete conversion, the mixture is cooled to 20° C. and then hydrolysed with water. The aqueous phase is removed and the organic phase is washed with aqueous sodium hydroxide solution. The organic phase is concentrated in vacuo before being purified by chromatography on a silica gel column using a toluene/ethanol mixture (93/7) as eluant. The elution solvent is then removed by concentration. The purified product is taken up in a mixture of toluene and methyl tert-butyl ether (MTBE) (w/w 35/65) at 20° C. The product is precipitated by adding that solution to a large excess of cyclohexane. The suspension is then filtered and then the cake is washed with cyclohexane. The product is dried with a temperature gradient from 20 to 40° C. to give N-[4-(benzyloxy)phenyl]-5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-1,2-dimethyl-1H-pyrrole-3-carboxamide in the form of a white solid with a yield of 75% (purity by HPLC≥96.0%).

### Method 2.

5-{5-Chloro-2-[(3S)-3-[(morpholin-4-yl)methyl]-3,4-dihydroisoquinoline-2(1H)-carbonyl]phenyl}-1,2-dimethyl-1H-pyrrole-3-carboxylic acid hydrochloride (1.000 kg) and 4-[4-(benzyloxy)anilino]-1,5-dimethyl-1H-pyrrole-2-carbonitrile (0.583 kg) are suspended in chlorobenzene (8.0 L) and then the mixture is heated to 120° C. Pyridine (0.581 kg) and 50% propylphosphonic anhydride in ethyl acetate (1.753 kg) are slowly added. After complete conversion, the mixture is cooled to 20° C. and then hydrolysed with water. The aqueous phase is removed and the organic phase is washed with aqueous sodium hydroxide solution (IN). The organic phase is concentrated in vacuo to 3L and finally diluted with 20L of ethyl acetate. N-[4-(benzyloxy)phenyl]-5-{5-chloro-2-[(3S)-3-[(morpholin-4-yl)methyl]-3,4-dihydroisoquinoline-2(1H)-carbonyl]phenyl }-N-(5-cyano-1,2-dimethyl-1H-pyrrol-3-yl)-1,2-dimethyl-1H-pyrrole-3-carboxamide is stored in solution until the next step with a theoretical yield of 100%.

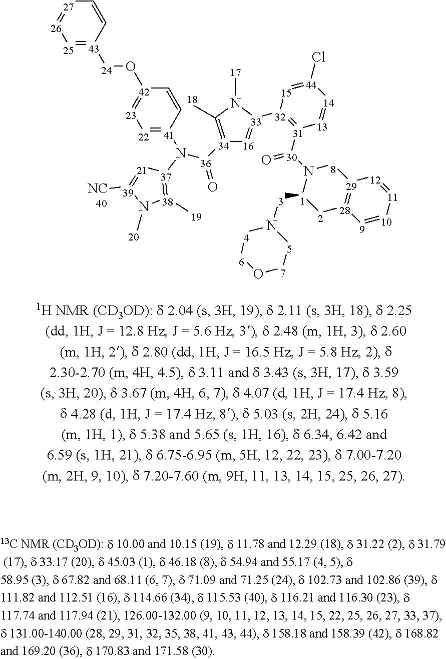

STEP 5—Preparation of 5-{5-chloro-2-1(3S)-3-[(morpholin-4-yl)methyl]-3,4-dihydroisoquinoline-2(1H)-carbonyl|phenyl}-N-(5-cyano-1,2-dimethyl-1H-pyrrol-3-yl)-N-(4-hydroxyphenyl)-1,2-dimethyl-1H-pyrrole-3-carboxamide

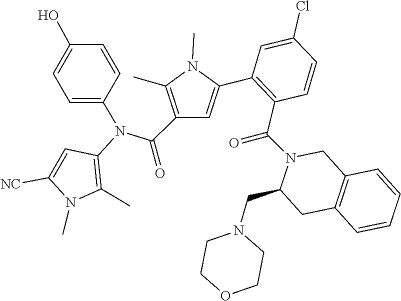 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

### Method 1

N-[4-(Benzyloxy)phenyl]-5-{5-chloro-2-[(3S)-3-[(morpholin-4-yl)methyl]-3,4-dihydroisoquinoline-2(1H)-carbonyl]phenyl }-N-(5-cyano-1,2-dimethyl-1H-pyrrol-3-yl)-1,2-dimethyl-1H-pyrrole-3-carboxamide (1.000 kg) obtained in Step 4 (Method 1) is dissolved in ethyl acetate (9.02 kg) at 25° C. and then a 33% solution of hydrobromic acid in acetic acid (2.800 kg) is added. The reaction mixture is maintained at 25° C. until conversion is complete. The mixture is hydrolysed with water and then the pH is adjusted to 8.5±0.5 by addition of 10N sodium hydroxide solution. After a contact time, the aqueous phase is counter extracted with ethyl acetate. The organic phases are combined and concentrated in vacuo. Then, the product is purified by chromatography on a silica gel column using a toluene/ethanol mixture (95/5) to (93/7) as eluant. The elution solvent is then removed by concentration to a residual volume of 3.5 L. 5-{5-Chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-N-(4-hydroxyphenyl)-1,2-dimethyl-1H-pyrrole-3-carboxamide is therefore obtained in solution in toluene with a yield of approximately 90% (purity by HPLC≥98.0%).

### Method 2

Acetyl chloride (77.8 g) is added to ethanol (1.0 L) and after 30 minutes, N-[4-(benzyloxy)phenyl]-5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-1,2-dimethyl-1H-pyrrole-3-carboxamide obtained in Step 4 (Method 1) (100 g) is added at 20° C. Palladium hydroxide on carbon 20% (10 g) is suspended and then the mixture is heated to 55° C. The deprotection is performed under atmospheric pressure with hydrogen. After complete conversion, the suspension is clarified at 20° C. and palladium is washed with ethanol (200 mL). The pH of the mother liquor is adjusted to 8 with a sodium hydroxide solution. A solvent swap from ethanol to ethyl acetate is carried out and the organic layer is washed with water (850 mL) and concentrated in vacuo before being purified by chromatography on a silica gel column using toluene/ethyl acetate mixture (95/5) to (93/7) as eluant. The elution solvent is then removed by concentration to give 5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-N-(4-hydroxyphenyl)-1,2-dimethyl-1H-pyrrole-3-carboxamide in the form of a pink solid with a yield of 80%.

### Method 3

To the solution of N-[4-(benzyloxy)phenyl]-5-{5-chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-1,2-dimethyl-1H-pyrrole-3-carboxamide (1.483 kg) in a mixture of chlorobenzene and ethyl acetate, obtained in Step 4 (Method 2), is added at 20° C. a 33% solution of hydrobromic acid in acetic acid (4.15 kg). The reaction mixture is maintained at 20° C. until conversion is complete. The mixture is hydrolysed with water and then a 10N sodium hydroxide solution (approximative quantity 8.3 kg). After a contact time, the aqueous phase is counter extracted with ethyl acetate. The organic phases are combined and concentrated in vacuo. Then, the product is purified by chromatography on a silica gel column using a toluene/ethanol mixture as eluant. The elution solvent is then removed by concentration to a residual volume of 3.5 L. 5-{5-Chloro-2-[(3S)-3-[(morpholin-4-yl)methyl]-3,4-dihydroisoquinoline-2(1H)-carbonyl]phenyl}-N-(5-cyano-1,2-dimethyl-1H-pyrrol-3-yl)-N-(4-hydroxyphenyl)-1,2-dimethyl-1H-pyrrole-3-carboxamide is therefore obtained in toluene with a yield of 85% (yield for two successive steps).

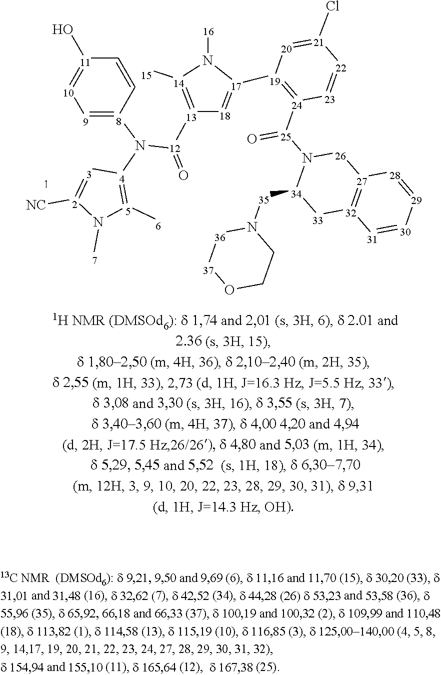

1-49 (canceled)

50. A process for preparing a compound of formula (V): 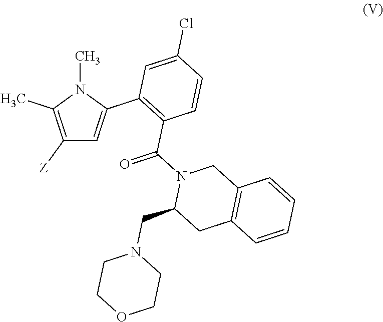
wherein:
Z represents —COOR or —CN, wherein
R represents a (C₁-C₆)alkyl group, an allyl group or a —CH₂-aryl group,
comprising the step of reacting a compound of formula (III): 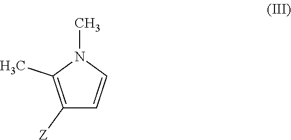
wherein Z is as defined above,
with a compound of formula (IV): 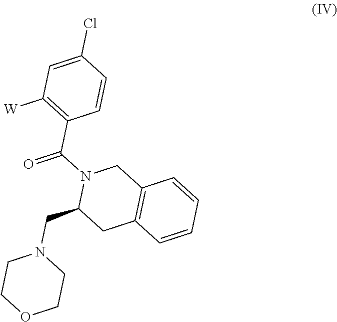
wherein W represents a leaving group selected from a halogen atom, a trifluoromethanesulfonate group, a methanesulfonate group and a para-toluenesulfonate group,
in a solvent or a mixture of solvents, at a temperature greater than 70° C. in the presence of:
(i) a palladium catalyst;
(ii) optionally a phosphine; and
(iii) a base.

51. The process according to claim 50, wherein Z is —COOR, wherein R represents a methyl, ethyl, isopropyl, tert-butyl, benzyl or a para-methoxybenzyl group.

52. The process according to claim 50, wherein W represents a bromine atom.

53. The process according to claim 50, wherein the palladium catalyst is palladium(II)acetate (Pd(OAc)₂).

54. The process according to claim 50, wherein the reaction mixture comprises a phosphine selected from tri-tert-butylphosphine, XPhos, CyJohnPhos and Tri(o-tolyl)-phosphine.

55. The process according to claim 50, wherein the solvent is an aprotic solvent.

56. The process according to claim 55, wherein the solvent is selected from dimethylsulfoxide (DMSO), N-butylpyrrolidinone (NBP), 2-methyltetrahydrofuran and toluene.

57. The process according to claim 50, wherein the temperature greater than 90° C.

58. The process according to claim 50, wherein the base is a carbonate salt.

59. The process according to claim 50, wherein the reaction mixture further comprises pivalic acid.

60. The process according to claim 50, wherein the compound of formula (IV), or an addition salt thereof with a pharmaceutically acceptable acid: 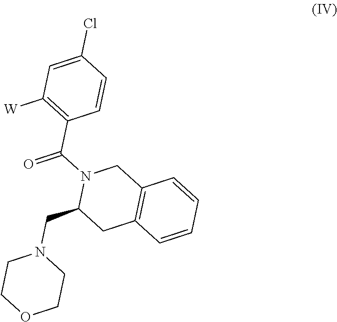
wherein W represents a leaving group selected from a halogen atom, a trifluoromethanesulfonate group, a methanesulfonate group and a para-toluenesulfonate group,
is obtained by a coupling reaction of a compound of formula (I): 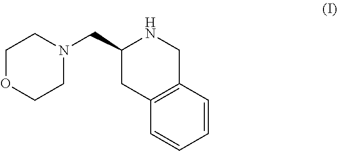 [SMILES: c1ccc2c(c1)CN[C@H](CN1CCOCC1)C2]
or an addition salt thereof with a pharmaceutically acceptable acid,
with a compound of formula (II): 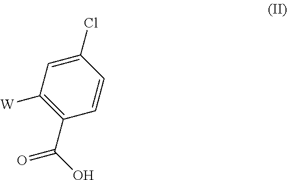
in an aprotic solvent in the presence of an amine base and a coupling agent.

61. The process according to claim 60, wherein the compound of formula (II) is 2-bromo-4-chlorobenzoic acid thus leading to the formation of a compound of formula (IV-a):

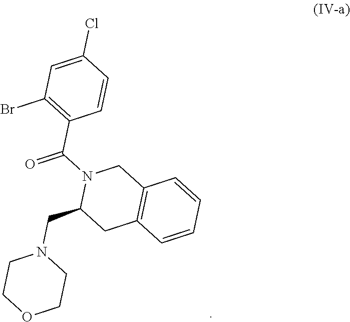 [SMILES: O=C(c1ccc(Cl)cc1Br)N1Cc2ccccc2C[C@H]1CN1CCOCC1]

62. The process according to claim 60, wherein the compound (I) is in the form of a dihydrochloride salt.

63. The process according to claim 60, wherein the coupling agent is selected from propylphosphonic anhydride, cyanuric chloride, methyl propiolate, tetraethyl orthosilicate, pivalyl chloride, N-ethoxycarbonyl-2-ethoxy-1,2-dihydroquinoline, isobutylchloroformate, thionyl chloride and oxalyl chloride.

64. The process according to claim 60, wherein the amine base is selected from triethylamine, N,N-diisopropylethylamine, 1,4-diazabicyclo[2.2.2]octane, 1,8-diazabicyclo[5.4.0]undec-7-ene, N-methylmorpholine, N-ethylmorpholine, pyridine and 2,6-lutidine.

65. The process according to claim 60, wherein the coupling reaction between the compound of formula (I) and the compound of formula (II) is carried out at a temperature between 20 to 50° C.

66. The process according to claim 60, wherein the aprotic solvent employed in the coupling reaction between the compound of formula (I) and the compound of formula (II) is selected from ethyl acetate, methylene chloride and isopropyl ether.

67. The process according to claim 60, wherein the compound of formula (IV) is isolated as a free base.

68. The process according to claim 50, wherein the ester or nitrile function of the compound of formula (V): 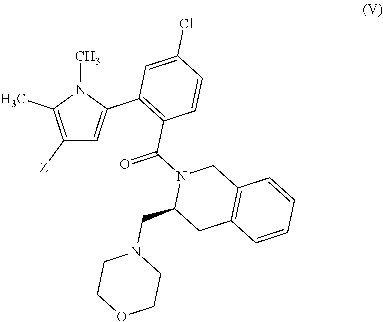
wherein: Z represents -COOR or -CN, wherein
R represents a (C₁-C₆)alkyl group, an allyl group or a —CH₂-aryl group, is further hydrolysed in a protic medium to yield a compound of formula (VI): 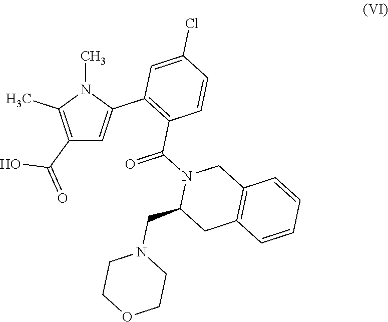 [SMILES: Cc1c(C(=O)O)cc(-c2cc(Cl)ccc2C(=O)N2Cc3ccccc3C[C@H]2CN2CCOCC2)n1C]
which compound of formula (VI) is isolated as a zwitterion or in the form of an addition salt thereof with a pharmaceutically acceptable acid, before being subjected to a peptidic coupling with 4-[4-(benzyloxy)anilino]-1,5-dimethyl-1H-pyrrole-2-carbonitrile of formula (VII): 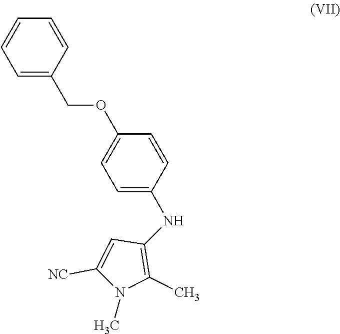 [SMILES: Cc1c(Nc2ccc(OCc3ccccc3)cc2)cc(C#N)n1C]
in an aprotic solvent in the presence of a coupling agent and optionally in the presence of an amine base,
to yield a compound of formula (VII): 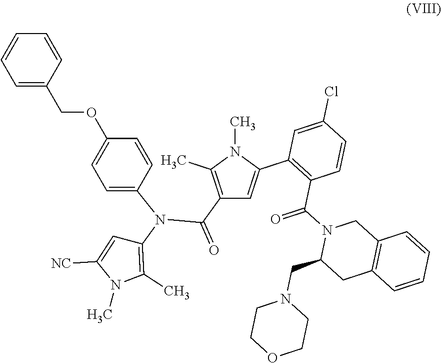 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]
which compound of formula (VII) is deprotected under acidic conditions to yield Compound A: 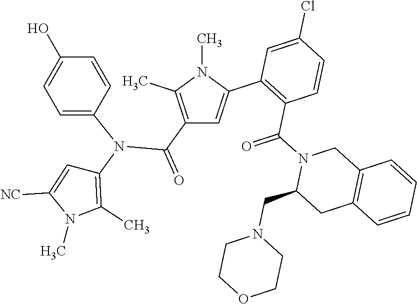 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]
which compound is isolated and may be converted into its addition salts with a pharmaceutically acceptable acid or base.

69. The process according to claim 68, wherein Z is —COOR, wherein R represents a methyl, ethyl, isopropyl, tert-butyl, benzyl or a para-methoxybenzyl group.

70. The process according to claim 68, wherein the compound of formula (V) is: 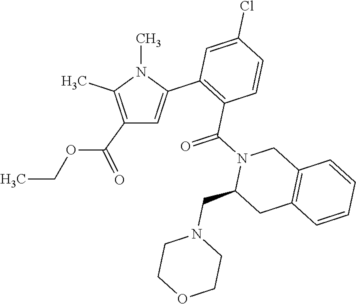 [SMILES: CCOC(=O)c1cc(-c2cc(Cl)ccc2C(=O)N2Cc3ccccc3C[C@H]2CN2CCOCC2)n(C)c1C]
which compound is hydrolysed under basic conditions.

71. The process according to claim 68, wherein the compound of formula (V) is: 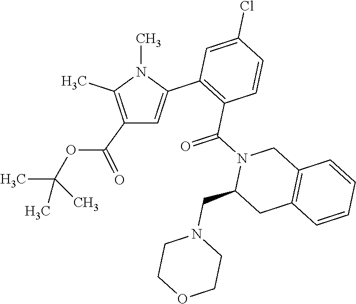 [SMILES: Cc1c(C(=O)OC(C)(C)C)cc(-c2cc(Cl)ccc2C(=O)N2Cc3ccccc3C[C@H]2CN2CCOCC2)n1C]
which compound is hydrolysed under acidic conditions.

72. The process according to claim 68, wherein the protic medium used for the hydrolysis of compound (V) is methanol, ethanol, isopropanol, DMSO/water or an ethanol/water mixture.

73. The process according to claim 72, wherein the protic medium is ethanol/water and the hydrolysis of the compound of formula (V) is carried out at a temperature between 60 and 80° C.

74. The process according to claim 68, wherein the compound of formula (VI) is isolated in the form of an addition salt with a pharmaceutically acceptable acid selected from hydrochloric acid, sulfuric acid, hydrobromic acid, para-toluenesulfonic acid, methanesulfonic acid, 1,5-naphtalenedisulfonic, acetic acid, trifluoroacetic acid, fumaric acid, tartric acid, oxalic acid, citric acid, succinic acid, maleic acid, phosphoric acid and boric acid.

75. The process according to claim 68, wherein the coupling agent is selected from thionyl chloride, isobutyl chloroformate, N-ethoxycarbonyl-2-ethoxy-1,2-dihydroquinoline and propylphosphonic anhydride.

76. The process according to claim 68, wherein the aprotic solvent used for the peptidic coupling is selected from dichloromethane, acetonitrile, toluene, ethyl acetate, butyl acetate, isobutyl acetate, propyl acetate, isopropyl acetate, chlorobenzene, N,N-dimethylformamide and pyridine.

77. The process according to claim 68, wherein the coupling agent is N-ethoxycarbonyl-2-ethoxy-1,2-dihydroquinoline and the solvent is toluene.

78. The process according to claim 68, wherein the peptidic coupling is carried out in the presence of an amine base.

79. The process according to claim 78, wherein the amine base is selected from pyridine, N,N-diisopropylethylamine and triethylamine.

80. The process according to claim 68, wherein the coupling agent is propylphosphonic anhydride and the peptidic coupling is carried out in the presence of an amine base which is pyridine.

81. The process according to claim 68, wherein the coupling agent is propylphosphonic anhydride, the peptidic coupling is carried out in the presence of an amine base which is pyridine and the aprotic solvent is selected from the group: acetonitrile, toluene, chlorobenzene, ethyl acetate, butyl acetate and propyl acetate.

82. The process according to claim 81, wherein the peptidic coupling is carried out at a temperature between 60 and 135° C.

83. The process according to claim 68, wherein the deprotection of the compound of formula (VIII) is carried out in the presence of hydrobromic acid, hydrochloric acid, sulfuric acid, methanesulfonic acid, a mixture of hydrochloric acid and acetic acid, or a mixture of hydrobromic acid and acetic acid.

84. The process according to claim 83, wherein the solvent employed for the deprotection of the compound of formula (VIII) is selected from dichloromethane, chlorobenzene, dioxane and ethyl acetate.

85. The process according to claim 83, wherein the temperature for the deprotection reaction is maintained below 40° C.

86. The process according to claim 68, wherein the deprotection of the compound of formula (VIII) is carried out via a hydrogenation reaction in the presence of a catalyst under acidic conditions.

87. The process according to claim 86, wherein:
the palladium catalyst is Pd(OH)₂ on carbon or palladium on carbon and
the hydrogenation reaction is carried out in hydrochloride ethanol at a temperature between 40 and 65° C.

88. A process for preparing Compound A: 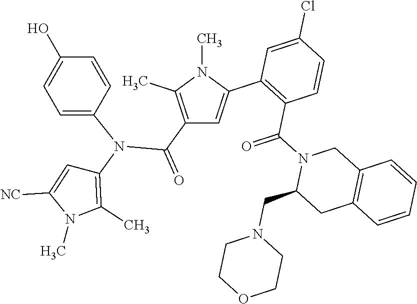 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]
wherein a compound of formula (I): 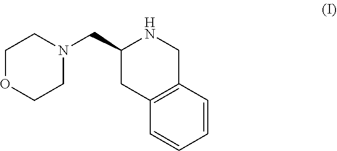 [SMILES: c1ccc2c(c1)CN[C@H](CN1CCOCC1)C2]
or an addition salt thereof with a pharmaceutically acceptable acid, is subjected to a coupling reaction with a compound of formula (II): 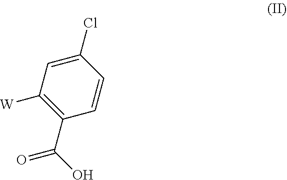
wherein W represents a leaving group selected from a halogen atom, a trifluoromethanesulfonate group, a methanesulfonate group and a para-toluenesulfonate group,
in an aprotic solvent in the presence of an amine base and a coupling agent at a temperature between 20 to 50° C.,
to yield a compound of formula (IV), or an addition salt thereof with a pharmaceutically acceptable acid: 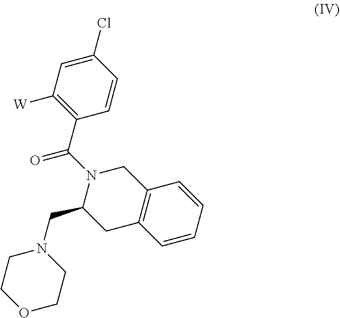
which compound of formula (IV) is reacted with a compound of formula (III): 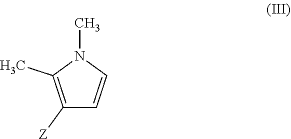
wherein:
Z represents —COOR or —CN, wherein
R represents a (C₁-C₆)alkyl group, an allyl group or a —CH₂-aryl group,
in a solvent or a mixture of solvents, at a temperature greater than 70° C. in the presence of:
(i) a palladium catalyst;
(ii) optionally a phosphine; and
(iii) a base,
to yield a compound of formula (V): 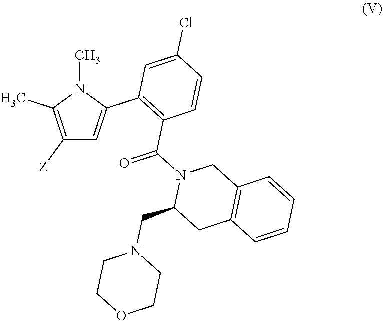
the ester or nitrile function of which compound of formula (V) is hydrolysed in a protic medium to yield the compound of formula (VI): 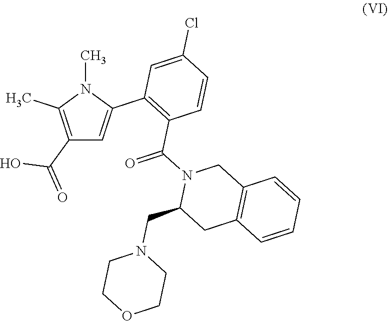 [SMILES: Cc1c(C(=O)O)cc(-c2cc(Cl)ccc2C(=O)N2Cc3ccccc3C[C@H]2CN2CCOCC2)n1C]
which compound of formula (VI) is isolated as a zwitterion or in the form of an addition salt thereof with a pharmaceutically acceptable acid, before being subjected to a peptidic coupling with 4-[4-(benzyloxy)anilino]-1,5-dimethyl-1H-pyrrole-2-carbonitrile of formula (VII): 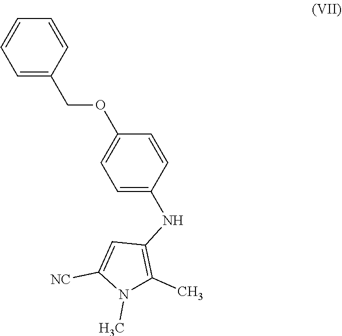 [SMILES: Cc1c(Nc2ccc(OCc3ccccc3)cc2)cc(C#N)n1C]
in an aprotic solvent in the presence of a coupling agent and optionally in the presence of an amine base,
to yield the compound of formula (VI): 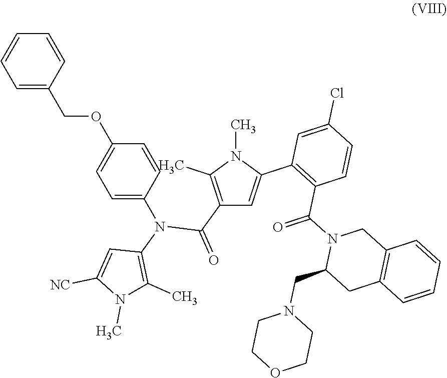 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]
which compound of formula (VIII) is deprotected under acidic conditions to yield the Compound A: 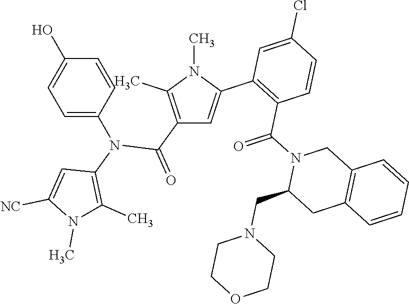 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]
which compound is isolated and may be converted into its addition salts with a pharmaceutically acceptable acid or base.

89. A compound of formula (IV), or an addition salt thereof with a pharmaceutically acceptable acid: 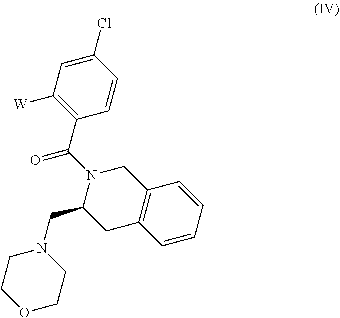
wherein W represents a leaving group selected from a halogen atom, a trifluoromethanesulfonate group, a methanesulfonate group and a para-toluenesulfonate group.

90. A compound of formula (V): 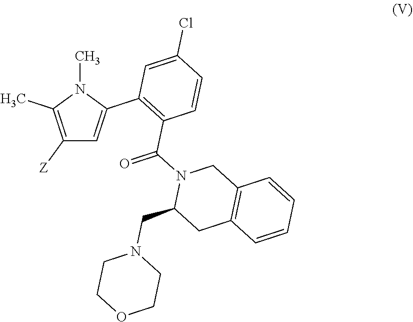
wherein: Z represents —COOR and —CN, wherein
R represents a (C1-Co)alkyl group, an allyl group or a —CH₂-aryl group, with the proviso that R does not represent an ethyl group.

91. A compound of formula (VII):

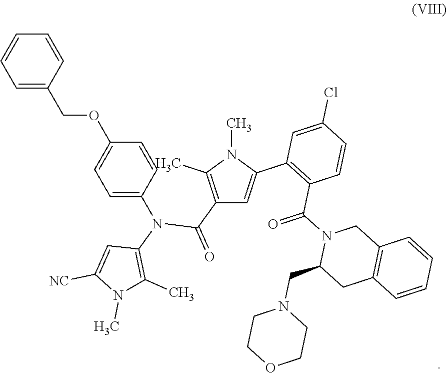 [SMILES: Cc1c(N(C(=O)c2cc(-c3cc(Cl)ccc3C(=O)N3Cc4ccccc4C[C@H]3CN3CCOCC3)n(C)c2C)c2ccc(OCc3ccccc3)cc2)cc(C#N)n1C]

92. A compound of formula (VII):

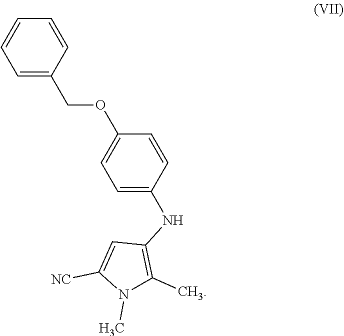 [SMILES: Cc1c(Nc2ccc(OCc3ccccc3)cc2)cc(C#N)n1C]

93. A process for the preparation of the compound of formula (VII) according to claim 92, comprising the step of reacting a compound of formula (SM1-VIII): 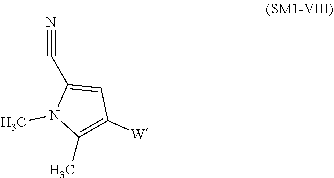
wherein W′ represents a leaving group selected from a halogen atom, a trifluoromethanesulfonate group, a methanesulfonate group and a para-toluenesulfonate group, with a compound of formula (SM2-VIII): 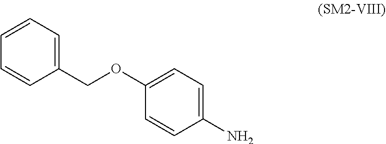 [SMILES: Nc1ccc(OCc2ccccc2)cc1]
in the presence of a palladium-phosphine complex catalyst and a base in a polar aprotic solvent at a temperature between 40 and 85° C.,
wherein the palladium-phosphine complex catalyst is ready for use or prepared in situ starting from a palladium catalyst and a phosphine.

94. The process according to claim 93, wherein W′ represents a bromine atom.

95. The process according to claim 93, wherein the solvent is selected from N,N-dimethylformamide, dimethylsulfoxide and 2-methyltetrahydrofuran.

96. The process according to claim 93, wherein the palladium-phosphine complex catalyst is selected from tBuXPhos Pd G1, tBuXPhos Pd G3, BrettPhos G3 and tBuXPhosPd(allyl)OTf.

97. The process according to claim 93, wherein the palladium-phosphine complex catalyst is prepared in situ starting from Pd₂dba₃ and tBuXPhos.

98. The process according to claim 93, wherein the base is selected from tBuONa. tBuOK, K₃PO₄ and K₂CO₃.
